(19) United States
(12) Patent Application Publication
(10) Pub. No.: US 2022/0081538 A1
(43) Pub. Date: Mar. 17, 2022

(54) TAILORED LAYERS OF CELLULOSE DISPERSIONS FOR DETECTING ANALYTES

(71) Applicant: Fraunhfer-Gesellschaft zur Förderung der angewandten Forschung e.V., München (DE)

(72) Inventors: Eva EHRENTREICH-FOERSTER, Nuthetal (DE); Cornelia HETTRICH, Schwielowsee (DE); Kay HETTRICH, Schwielowsee (DE)

(21) Appl. No.: 17/279,064

(22) PCT Filed: Sep. 24, 2019
(86) PCT No.: PCT/EP2019/075698

§ 371 Date: Mar. 23, 2021

(30) Foreign Application Priority Data
Sep. 24, 2018 (DE) 10 2018 007 556.8

(51) Int. Cl. C08L 1/02; C09D 101/02; G01N 33/548

(52) U.S. Cl. CPC C08L 1/02; C09D 101/02; G01N 33/548; A61B 5/082; C08L 2203/02; G01N 2400/26; C08L 2205/025

(57) ABSTRACT

A process for producing a cellulose layer for the detection of at least one analyte includes producing a cellulose layer by applying a stable dispersion of cellulose and/or a cellulose derivative to a suitable support, and immobilizing at least one ligand on the cellulose layer. A cellulose layer produced by the process can be employed in detection methods, devices, kits, and uses.

### PRIORITY AND CROSS REFERENCE TO RELATED APPLICATIONS

This application is the U.S. National Phase Application under 35 U.S.C. § 371 of International Application No. PCT/EP2019/075698, filed Sep. 24, 2019, designating the U.S. and published as WO 2020/064723 A1 on Apr. 2, 2020, which claims the benefit of German Application No. DE 10 2018 007 556.8, filed Sep. 24, 2018. Any and all applications for which a foreign or a domestic priority is claimed is/are identified in the Application Data Sheet filed herewith and is/are hereby incorporated by reference in their entireties under 37 C.F.R. § 1.57.

### FIELD

The present invention relates to tailored layers of cellulose dispersions for detecting analytes.

### BACKGROUND

Cellulose is an economically important natural material and is used inter alia as building material, for paper manufacture, for clothing, and in the energy industry.

### SUMMARY

The present invention relates to a process for producing a cellulose layer for the detection of at least one analyte, comprising (i) producing a cellulose layer by applying a stable dispersion of cellulose and/or a cellulose derivative to a suitable support, and (ii) immobilizing at least one ligand on the cellulose layer; and also relates to cellulose layer produced by said process. The present invention additionally relates to associated detection methods, devices, kits, and uses.

### BRIEF DESCRIPTION OF THE DRAWINGS

FIG. 1: Comparison of the signal-background (BG) intensities of peptides (C1-C2) and proteins (wtC) on cellulose coating (excitation at 635 nm); small bars for background signals (using slide S1 D3)

FIG. 2: Comparison of the signal-background intensities of peptides (C1-C2) and proteins (wtC) on (in-house) epoxy slide (excitation at 635 nm)

FIG. 3: Schematic representation of a support with coating; A) one functionality, B) plurality of functionalities.

FIG. 4: A) Exemplary representation of a coated support; B) Schematic representation of the structure of cellulose and of substitution options.

FIG. 5: A) Schematic exemplary representation of the production of layers of the invention; B) Examples for the transparency properties of the cellulose layers of the invention.

FIG. 6: Immobilization of ligands (exemplary and schematic) FIG. 7: Example for a support coated with a cellulose layer of the invention

FIG. 8: Spot morphology, comparison of cellulose and (in-house) epoxy slide coating

FIG. 9: Stress test: Surface; comparison of different cellulose layer thicknesses, in-house epoxy coating

FIG. 10: Immobilization and detection of proteins and peptides on a support; detection at 635 nm.

### DETAILED DESCRIPTION

Cellulose is an economically important natural material and is used inter alia as building material, for paper manufacture, for clothing, and in the energy industry. In recent years, modifications of cellulose have been developed that enable new applications. For example, cellulose materials having dimensions in the nanometer range have been developed, which are generally referred to as nanocelluloses. Nanocelluloses can be produced by different processes and from different starting materials. A distinction is generally made between the types shown in Table 1.

TABLE 1
Types of nanocellulose (after Klemm et al. (2011),
Angew. Chemie Int. Ed., 50: 5438-66)

| Type | Synonyms | Dimensions |
|---|---|---|
| Microfibrillated | Nanofibrillated | Diameter: 5-60 nm |
| cellulose | cellulose | Length: a few μm |
| (MFC) | (NFC) | |
| Nanocrystalline | Cellulose | Diameter: 5-70 nm |
| cellulose | nanocrystals, | Length: 100-250 nm |
| (NCC) | microcrystals, whiskers, | |
| | rod-shaped cellulose | |
| Bacterial | Bacterial cellulose, | Diameter: 20-100 nm |
| nanocellulose | microbial cellulose, | Nanofiber network |
| (BNC) | biocellulose | |

Mixed layers with nanocellulose and e.g. acrylic polymers have likewise been suggested (Grüneberger et al. (2014), J Mater Sci 49: 6437). On account of their favorable biological, chemical, and physical properties, nanocellulose materials have also been proposed for use in biomedical sciences, for example as a scaffold material or as a support material for drugs.

In medicine, biotechnology, agriculture, food science, and environmental science, there are many challenges, the solutions for which would be massively aided by the rapid detection of particular analytical parameters. Such tests are now established in many areas of life. The best-known examples are pH paper, pregnancy tests, or the determination of water hardness. All said examples convey the relevant information by means of a simple color change. Such test strips are made up of a support (plastic, paper, glass), an indicator (organic dye), and one or more polymer layers to fix the indicator.

Signals can in principle be generated in different ways: spectroscopically (e.g. fluorescence, luminescence, IR, UV); electrochemically (e.g. by amperometry, potentiometry, conductometry, coulometry); or label-free optical surface analysis (e.g. ellipsometry, reflectometric interference spectroscopy, surface plasmon resonance).

Technologies for the execution of multiparameter or even multiplex applications are playing an increasingly important role. Multiparameter analyses enable the simultaneous determination of a plurality of analytes in one measurement run and hence provide more complex analytical information after just one laboratory investigation.

Biochips increasingly dominate such detection techniques on account of their ability to perform a highly parallel measurement of many analytes with limited sample volumes. By virtue of its substantial miniaturization, the technology provides the basis for performing reactions based on biomolecules such as DNA, peptides or proteins. These are immobilized for this purpose on a support (chip) in a fixed grid. In general, the biomolecules are dissolved in aqueous liquids, which are applied to the solid substrate in the form of tiny droplets. For the production of biochips for multiparameter analyses, a surface with optimal chemical functions is a fundamental prerequisite. Appropriate surface properties enable the specific immobilization of different biomolecules and the generation of selective properties in hydrophilic or hydrophobic surfaces. Another problem with the existing market-relevant solutions is that it is possible to use only one specific biochip for each analytical task.

There is therefore a need for reliable means of producing coated surfaces, in particular surfaces having optically favorable properties and surfaces that allow further derivatization without interfering with subsequent uses.

This problem is solved by the processes/methods, cellulose layers, and uses having the features of the independent claims. Preferred embodiments, which can be put into practice in isolation or in any combination, are given in the dependent claims.

The present invention accordingly relates to a process for producing a cellulose layer for the detection of at least one analyte, comprising (i) producing a cellulose layer by applying a stable dispersion of cellulose and/or a cellulose derivative to a suitable support, and (ii) immobilizing at least one ligand on the cellulose layer.

In the following text, the terms “have”, “comprise” or “include” and any grammatical variations thereof are preferably used in a non-exclusive manner. These terms can therefore refer both to a situation in which, in addition to the features introduced by the terms, there are no further features in the object described, and to a situation in which one or more further features are present. For example, the wordings “A includes B”, “A comprises B”, and “A has B” refer not only to a situation in which no further element is present in A other than B, that is to say to a situation in which A consists exclusively of B, but also to a situation in which, in addition to B, one or more further elements are present in A, for example element C, elements C and D, or even further elements.

Furthermore, the terms “preferably”, “more preferably”, “most preferably”, “in particular”, “specifically” or similar wordings are hereinbelow used preferably in connection with optional features, without restricting further possibilities. Features introduced by these formulations are therefore preferably optional features and do not restrict the subject matter claimed in the claims. As will be understood by those skilled in the art, the invention can be executed with alternative features. The same applies to the wording “in an/one embodiment” or similar wordings that also refer to optional features without restriction in respect of further embodiments, without restricting the subject matter of the invention, and without restricting the possibility of combining the features thus introduced with other optional or non-optional features.

The term “standard conditions”, unless otherwise defined, refers to the IUPAC standard ambient temperature and pressure conditions (SATP), that is to say preferably a temperature of 25° C. and an absolute pressure of 100 kilopascals; standard conditions preferably additionally relate to a pH of 7. The term “approximately”, unless stated otherwise, refers to the specified value having the generally accepted technical precision in the relevant field of work and preferably to the specified value ±20%, preferably ±10%, even more preferably ±5%. The term “essentially” preferably refers to the fact that there are no possible deviations that have an influence on the stated result or on the use, i.e. potential deviations give rise to a deviation from the stated result of not more than ±20%, preferably ±10%, more preferably ±5%. “Consisting essentially of” therefore preferably means the presence of the specified constituents to the exclusion of other components with the exception of impurities, constituents that are unavoidable as a consequence of the production process, and/or constituents that have been added for a purpose that does not relate to the technical effect of the present invention. A composition that is defined by the phrase “consisting essentially of” can therefore contain additives, auxiliaries, solvents, diluents, support materials, and the like. A composition that should essentially consist of the specified components preferably contains not more than a mass fraction of 5%, preferably not more than 2%, more preferably not more than 1%, of components not specified.

The process of the invention can additionally comprise further steps; such further steps can relate for example to the production of a stable dispersion before application or to further steps following application, such as drying of the cellulose layer. Individual steps or all steps can be repeated; for example, a plurality of ligands can be applied in different application processes. The cellulose layer of the invention can remain on the support or, if in the form e.g. of a film or strip, can be removed therefrom after a drying process. In order to achieve different functionalities on the layer, aqueous dispersions of differently modified celluloses are preferably first produced. Two or more stable cellulose dispersions having the desired concentration ratios are then preferably first mixed with one another intensively. This mixture is then preferably applied as a layer to a desired support. Layers can thus be produced from different dispersions.

The term “support” is used in the context of the present description in the meaning familiar to those skilled in the art; the support is preferably an object or a device that is preferably rigid or flexible and that can in principle consist of any material. The support is preferably planar, cylindrical or ellipsoidal in shape, more preferably the support is a solid body in the form of a plate, film, pipe, membrane, or one or more beads. In particular for the production of a film or a strip, the stable dispersion can also be applied to, for example, a roll and dried there. The support can likewise preferably be a packaging material, a laboratory material, and/or a single-use article. Preferred packaging materials are films, made for example of polyethylene, polypropylene, polyvinyl chloride, or similar plastics. Preferred laboratory materials are supports for a biochip, for example microscope slides or similar materials, multiwell plates such as microtiter plates, semiconductor plates, or similar articles. Examples of preferred single-use articles are urine cups, syringes, cannulas, tubing, tissue articles, swabs, breathing masks or parts thereof, or air filters. The support preferably comprises glass, paper, plastic, ceramic, and/or metal, even more preferably the support consists of glass, paper, plastic, ceramic, and/or metal.

In a preferred embodiment, the support is transparent. In a further preferred embodiment, the cellulose layer is transparent. In a particularly preferred embodiment, the support and the cellulose layer are transparent. The term “transparent” is used in the context of the present description in the meaning familiar to those skilled in the art; the term transparent preferably refers to the property of a material of essentially not absorbing radiation, preferably visible light. Preferably, wavelengths in a range between 300 nm and 700 nm are essentially not absorbed, more preferably in a range between 350 nm and 650 nm. The absorption coefficient of a transparent material is preferably not more than 10 cm⁻¹, more preferably not more than 2 cm⁻¹, even more preferably not more than 1 cm⁻¹.

The term “cellulose” is known to those skilled in the art and refers to an organic polymer composed of glucose units connected by beta-1,4-glycosidic linkages. The production of cellulose is likewise known to those skilled in the art. Cellulose is preferably obtained from wood, annual plants, cotton, and/or waste paper. Preference is also given to using a derivative of cellulose; the derivatization introduced is preferably in the form of ester and/or ether groups, in particular one or more functional group(s) selected from carboxyl, carbonyl, sulfate, carboxymethyl, methyl, ethyl, silyl, acetyl, carbamate, and amino. The degree of substitution (DS), that is to say the average number of substituted hydroxy groups per glucose unit, is preferably not more than 1, even more preferably not more than 0.5. The cellulose is preferably a nanocellulose or a derivative thereof.

The term “analyte” is used in the context of the present description in the meaning familiar to those skilled in the art; the analyte is preferably a chemical substance, preferably a substance soluble in a solvent, preferably water. The analyte is preferably a low- or high-molecular-weight metabolite of a cell, of a tissue, of an organ, or of a body, or a substance that is used to change the chemical composition of a cell, of a tissue, of an organ or of a body. Preferred low-molecular-weight analytes are those that are used in medical diagnostics, thus particularly analytes for which a changed concentration in a body tissue or in a body fluid indicates a pathology. Preferred low-molecular-weight analytes are therefore glucose, hormones, in particular estrogens, lipids, in particular cholesterol, uric acid, ammonia, and the like. Preferred macromolecular analytes are in particular polypeptides and polynucleotides. Particularly preferred polypeptides include antibodies, glycoproteins, and phosphoproteins. Preference is also given to autoantigens, allergens, and also cells or cell constituents, for example constituents of bacterial cell envelopes or of viral particles. Particular preference is given to analytes, antibodies or antigens, preferably antigens that bind to antibodies.

The term “ligand” is used in the context of the present description in the meaning familiar to those skilled in the art; the ligand is preferably a chemical substance that binds, preferably specifically, to an analyte. Specific binding is preferably present when the binding of the ligand to the analyte is at least 5 times, preferably at least 10 times, even more preferably at least 100 times, most preferably at least 1000 times, as strong as it is to a substance that is not the analyte; the affinity being preferably expressed as dissociation constants of the corresponding complexes. Alternatively, specificity can also be determined by determining the signal-to-background ratio, the signal-to-background ratio in the case of specific binding being preferably at least 3, more preferably at least 10, even more preferably at least 100, most preferably at least 1000. Appropriate methods are known to those skilled in the art. The specificity is preferably a group specificity, that is to say a specificity for a group of non-identical molecules having at least one common structural feature; a corresponding group is, for example, that of the IgG molecules. More preferably, the specificity is a specificity for a specific chemical substance, e.g. for a polypeptide. The affinity of the ligand for the analyte is preferably high enough to enable detection of the analyte in the planned detection procedure. The dissociation constant K_{d} of the ligand/analyte complex is preferably not more than 10⁻³ M, more preferably not more than 10⁻⁴ M, even more preferably not more than 10⁻⁶ M, most preferably not more than 10⁻⁸ M. The ligand is preferably a polypeptide, a polynucleotide, a carbohydrate, or a fat. Even more preferably, the ligand is an antibody, a hormone, a glycolipid, a phospholipid, a glycoprotein, or a phosphoprotein. The ligand is also preferably a recombinant protein, a native protein, an autoantigen, an allergen and/or a cell or cell constituent, for example a constituent of bacterial cell envelopes or of viral particles.

The term “immobilize” is used in the context of the present description in the meaning familiar to those skilled in the art; immobilization preferably results in the ligand remaining essentially bound to the cellulose layer during use. Preferably, a maximum of 10%, preferably a maximum of 2%, even more preferably a maximum of 1%, of an immobilized ligand leaches into a surrounding solution under standard conditions over a period of one hour. The ligand is preferably able to penetrate at least partially into the cellulose layer during the immobilization process; the term “immobilization on” a cellulose layer therefore preferably includes at least partial immobilization in the cellulose layer. The ligand is preferably immobilized on or in the cellulose layer non-covalently; the binding of the ligand to the cellulose layer is therefore preferably based on hydrogen bonds, van der Waals forces, and/or ionic interactions. The strength of immobilization can be controlled in particular through the use of suitable cellulose derivatives; for example, those skilled in the art may favor cationic cellulose derivatives for immobilizing anionic ligands, but more hydrophobic cellulose derivatives for binding hydrophobic ligands. In one embodiment, the ligand is covalently bonded to the cellulose layer; suitable reagents and side groups are known to those skilled in the art.

A multiplicity of non-identical ligands is preferably immobilized on the cellulose layer, the term “multiplicity” preferably referring to a number of at least two, even more preferably at least five, even more preferably at least ten, most preferably at least 20, non-identical ligands. The term “multiplicity” likewise preferably refers to a number of 2 to 15, preferably 2 to 10, particularly preferably 1 to 6, non-identical ligands. The non-identical ligands can in principle be immobilized as a mixture; they are preferably applied and immobilized on or in the cellulose layer separately, even more preferably in a spatially structured arrangement. The cellulose layer for the detection of at least one analyte thus preferably comprises a spatially structured arrangement (“array”) of ligands that preferably permits identification of the position of application of the individual ligands.

The term “dispersion” is known to those skilled in the art as a term for a heterogeneous mixture of at least two substances. The dispersion is preferably a liquid/solid dispersion, that is to say a suspension. The mass fraction in the dispersion is preferably within a range between 0.01% and 10%, more preferably between 0.05% and 5%. The mass fraction of cellulose and/or cellulose derivative in the dispersion is even more preferably between 0.01% and 10%, even more preferably between 0.05% and 5%. The dispersion medium is preferably an aqueous solution, more preferably water.

The term “stable dispersion” is used in the context of the present description in the meaning known to those skilled in the art and preferably refers to a dispersion in which the degree of dispersion remains essentially unchanged over a period of at least one month, preferably at least one year, even more preferably at least three years. Stable dispersion means that flocculation, aggregation or sedimentation preferably occurs only to a negligible extent in said period. The cellulose and/or the cellulose derivative in the stable dispersion preferably has a particle size of not more than 1000 nm, more preferably 750 nm, most preferably 600 nm, even more preferably all ingredients of the stable dispersion have a particle size of not more than 1000 nm, more preferably 750 nm, most preferably of not more than 600 nm. Methods for determining particle size are familiar to those skilled in the art; the particle size is preferably determined as described in the examples of the present description. The stable dispersion is preferably a dispersion of nanocellulose. The expression “applying a stable dispersion of cellulose and/or a cellulose derivative” is therefore preferably equivalent to the expression “applying a stable dispersion of nanocellulose and/or a nanocellulose derivative”. Methods for producing stable cellulose dispersions are known to those skilled in the art. The stable cellulose dispersion is preferably produced by high-pressure homogenization, as specified for example in DE 2009021688 and in WO 2009/021687. Stable cellulose dispersions are likewise preferably obtained by treatment in an Ultra-Turrax at approx. 20 000 revolutions/min for 15 minutes and preferably subsequent two-stage treatment in a high-pressure homogenizer. The treatment in the high-pressure homogenizer preferably includes six cycles in a 200 μm cell at 500 bar and preferably includes twelve cycles in a 50 μm cell at 1000 bar.

The cellulose layer can be applied by any method deemed appropriate by those skilled in the art; it is preferably applied by knife-coating, spraying, spin-coating, spray-drying, and/or dipping. The cellulose dispersion is preferably applied homogeneously. The layer thickness of the cellulose layer after drying is preferably from 0.01 μm to 10 μm, more preferably from 0.02 μm to 5 μm, even more preferably not more than 2.5 μm. Those skilled in the art will know that the layer thickness can be controlled not only through the choice of the application method, but in particular through the selection of the application volume and of the cellulose content of the dispersion. Examples for the realization of exemplary layer thicknesses are shown in particular in the examples.

The cellulose layer is preferably dried after application. The drying is carried out preferably at a temperature between 15° and 100°, more preferably between 30° and 80°, even more preferably between 35° and 65°. The drying is preferably carried out until the layer thickness is constant and/or to constant weight. Suitable drying processes are known to those skilled in the art. Preferred drying times are essentially determined by the application volume and the drying temperature. The immobilization of the ligand can preferably already occur during the drying of the cellulose layer, for example by admixing the ligand with the stable dispersion. More preferably, the ligand is immobilized on the dried or predried cellulose layer, for example by locally delimited application of small volumes of one or more ligand solution(s) (“spotting”). After immobilization, the cellulose layer is preferably dried again or used immediately. The cellulose layer of the invention is preferably not activated prior to further use. The cellulose layer of the invention is therefore prior to further use preferably not modified with chemical side chains that form covalent bonds with ligands, in particular the ligands described herein.

In the investigations underlying the present invention, it was surprisingly found that the particular structure of the cellulose layer of the invention and possibility of functionalization with chemical groups allow ligands from different groups of substances to be bound. A coating accordingly allows the achievement of various objects, for example in peptide, DNA or protein analyses. Starting from a given support, physical and chemical processes are used to create a tailored cellulose coating, which can if desired also be spatially resolved and multifunctional, that enables the parallel analysis even of different species. The adjustment of the surface properties leaves the general properties of the bulk material and of the support untouched.

For heterogeneous detection methods, in particular for use as biosensors, the surfaces can be functionalized with biomolecular probes or receptors. The multifunctional layers that are formed can be used in a wide variety of applications depending on the nature of this functionalization. Possible probe molecules are DNA for the investigation of pathologies or for determining the identity of sample material, antibodies for the detection of antigens, and antigens or antigen fragments for the serological detection of antibodies in biological samples. The substances to be applied can be immobilized on the multifunctional layers selectively and covalently.

The development of such functional materials for multiparameter analysis for “on-site tests” both in the field of quality and safety of food and animal feeds and for “point-of-need” diagnostics has a great many advantages, especially since the production conditions of the functional material can be adjusted so that the desired properties such as network density, functionality, and concentration meet the requirements of the immobilized probes. The advantages of the invention over the prior art consist in particular of the following:
modifiability and thus ability to create different functional groups on the surface, providing easy access to surface functionalities for peptides, proteins, DNA, and antibodies;
thin stable layers can be created by various methods;
very low background signal;
can be evaluated at different wavelengths;
top view and through vew possible;
no activation chemistry necessary.

Said advantages give rise to various options for application in everyday problems. Examples thereof are narrowing the analysis (diagnosis) in a particular pathology through the simultaneous use of different analytes/markers, and the performance of a relatively large number of tests from a large, heterogeneous area.

The definitions given above apply mutatis mutandis also to the embodiments described below.

The present invention also relates to a cellulose layer having an immobilized ligand, produced or producible by the process for producing a cellulose layer of the present invention.

The present invention additionally relates to a method for the detection of an analyte in a sample, comprising contacting the sample with a cellulose layer produced by the process for producing a cellulose layer of the present invention and/or with a cellulose layer of the present invention, and detection of analytes interacting with the ligands present in the cellulose layer.

The method for the detection of an analyte is preferably an in-vitro method and can additionally comprise further steps. Further steps can relate e.g. to obtaining a sample and/or to adding (further) reactants to a detection reaction. One or more steps of the method can also be executed in an automated process.

The term “contacting” is used in the context of the present description in the meaning known to those skilled in the art; contacting preferably comprises applying a liquid sample to the cellulose layer of the invention and enabling an interaction between ligand and analyte potentially present in the sample. In the case of a gaseous sample, the above applies mutatis mutandis; in this case, the cellulose layer is preferably wetted or preswollen. In the case of a solid sample, contacting can be accomplished e.g. by bringing the surface of the sample into contact with the cellulose layer, for example by laying one on top of the other.

The term “sample” is familiar to those skilled in the art and includes all sample materials that can potentially contain an analyte. The sample may be the whole object undergoing investigation, for example when investigating foods. The sample is preferably part of the object undergoing investigation. Preferred sample materials are liquid or gaseous samples; solid samples are preferably extracted with a suitable extraction liquid and then used in the same way as liquid samples. The samples are preferably pretreated, for example in order to detach the analyte from bonds or complexes or in order to remove possibly interfering sample constituents; even more preferably, the sample is not pretreated before contacting with the cellulose layer. The sample is preferably a biological sample, in particular a food or a sample collected for diagnostic purposes. The sample is preferably a tissue sample from a living organism, preferably a mammal, more preferably a human. Solid samples are preferably tissue samples or stool. More preferably, the sample is a gaseous sample, for example a breath sample, in particular an exhaled air sample. Even more preferably, the sample is a sample of a body fluid, preferably blood, plasma, serum, saliva, urine, cerebrospinal fluid, pleural fluid, ascites fluid, bile, sweat, mother's milk, menstrual fluid, ejaculate, smear material, in particular from the nose, mouth, or other mucous membranes, or lavage fluid from a bodily orifice; most preferably, the sample is blood, serum, plasma or urine. The sample is likewise preferably a sample matrix from environmental science or life sciences, in particular freshwater and drinking water, process water and waste water, soil, air or exhaust air.

Detection of the interaction of the ligand with the analyte preferably takes place by methods known to those skilled in the art, which are selected by those skilled in the art in accordance with requirements arising in particular from the sample material, identity of the analyte, and identity of the ligand. In the case of a polypeptide as analyte and an antibody as ligand, detection can take place e.g. by means of a secondary antibody that is coupled to a detectable chemical moiety such as a dye or an enzyme. In the case of a low-molecular-weight analyte, the ligand may for example be an enzyme that uses the analyte as a substrate. For detection, it may accordingly be necessary to add additional reactants, buffers, ions and the like in order to obtain a detectable reaction. Appropriate methods are known to those skilled in the art. Detection takes place preferably visually or by means of fluorescence optics, luminescence optics or absorption optics, by scanning densitometry or electrochemically. More preferably, detection takes place by imaging with fluorescence optics, luminescence optics, or absorption optics or by scanning densitometry.

The present invention also relates to a device comprising a cellulose layer of the invention.

The term “device” is used in the context of the present description in the meaning known to those skilled in the art; the device is preferably a device for determining an analyte in a sample or a part thereof, for example a probe or a test strip. The device preferably includes the cellulose layer of the invention in the form of a membrane, film, or in another suitable form. More preferably, the device includes the cellulose layer of the invention on a support. The device is therefore preferably a packaging material, a laboratory material, preferably a biochip or a multiwell plate, or a single-use article, preferably a urine cup, a syringe, a cannula, tubing, a tissue article, a swab, a breathing mask or a part thereof, or an air filter.

The present invention also relates to a kit for the detection of at least one analyte, comprising at least one cellulose layer having at least one immobilized ligand and a device for sample collection, the cellulose layer preferably being present on a support.

The term “kit” is used in the context of the present description in the meaning known to those skilled in the art; the term preferably refers to a combination of the specified components that is preferably tailored to enable detection of at least one analyte in a sample. The components can be packed together or individually. The kit is preferably configured for the performance according to the present invention of the method for detection of an analyte in a sample. The components are preferably provided ready-to-use. The kit preferably comprises further components, for example buffers, wash solutions, one or more detection reagents, and/or optionally instructions for use. In the kit of the invention, the cellulose layer is preferably fixed on a support, in particular on a plate, film, membrane or a bead. The cellulose layer is likewise preferably present on a biochip, a multiwell plate, a packaging material, or a single-use article, in particular on a urine cup, a syringe, a cannula, tubing, a tissue article, a swab, a breathing mask or part thereof, or an air filter.

The term “sample collection device” refers to any device that is suitable or configured for collecting a sample as specified above. Those skilled in the art will know which devices are suitable for intended sample collection in the individual case. Swabs, scalpels, punches, ventilation cannulas or tubing are preferred for collecting biological samples. Even more preferable as sample collection devices are syringes and/or cannulas. In the field of chemical and environmental analysis, the sample collection device is preferably a pipette, a swab, a spoon, a spatula or especially a single-use pipette.

The present invention further relates to the use of a cellulose layer produced according to the process of the present invention for the detection of an analyte, preferably in medical diagnostics, (bio)analysis, environmental analysis, the agricultural, foodstuffs or packaging industry, process engineering or forensic medicine.

In light of the above, the following embodiments are contemplated in particular:

Embodiment 1: A process for producing a cellulose layer for the detection of at least one analyte, comprising

(i) producing a cellulose layer by applying a stable dispersion of cellulose and/or a cellulose derivative to a suitable support, and

(ii) immobilizing at least one ligand on the cellulose layer.

Embodiment 2: The process according to embodiment 1, wherein at least two non-identical celluloses and/or cellulose derivatives are dispersed together before application.

Embodiment 3: The process according to embodiment 1 or 2, wherein the cellulose layer is present on a support, preferably on an essentially transparent support, more preferably on a transparent support.

Embodiment 4: The process according to any of embodiments 1 to 3, wherein the ligand is a compound having an affinity for the at least one analyte.

Embodiment 5: The process according to any of embodiments 1 to 4, wherein the ligand selectively binds the at least one analyte.

Embodiment 6: The process according to any of embodiments 1 to 5, wherein the ligand is a polypeptide, a polynucleotide, a carbohydrate, or a fat.

Embodiment 7: The process according to any of embodiments 1 to 6, wherein the ligand is an antibody, a hormone, a glycolipid, a phospholipid, a glycoprotein, or a phosphoprotein.

Embodiment 8: The process according to any of embodiments 1 to 7, wherein the ligand is or comprises a recombinant protein, a native protein, an autoantigen, an allergen and/or a cell.

Embodiment 9: The process according to any of embodiments 1 to 8, wherein a multiplicity of non-identical ligands is immobilized on the cellulose layer, said ligands preferably having affinities for non-identical analytes.

Embodiment 10: The process according to any of embodiments 1 to 9, wherein immobilization takes place in a spatially structured manner.

Embodiment 11: The process according to any of embodiments 1 to 10, wherein the coated support is configured for visual evaluation, or evaluation by imaging with fluorescence optics, luminescence optics or absorption optics, evaluation by scanning densitometry and/or electrochemical evaluation.

Embodiment 12: The process according to any of embodiments 1 to 11, wherein the ligand is covalently bonded to the cellulose layer.

Embodiment 13: The process according to any of embodiments 1 to 12, wherein the cellulose layer obtained is essentially transparent, preferably wherein the cellulose layer obtained is transparent.

Embodiment 14: The process according to any of embodiments 1 to 13, wherein the stable dispersion has a solids content (mass fraction) of between 0.05% (w/w) and 5% (w/w), preferably a content of cellulose and/or cellulose derivative of between 0.05% (w/w) and 5% (w/w).

Embodiment 15: The process according to any of embodiments 1 to 14, wherein the cellulose and/or the cellulose derivative have a particle size of not more than 600 nm, preferably wherein the ingredients of the stable dispersion have a particle size of not more than 600 nm.

Embodiment 16: The process according to any of embodiments 1 to 15, wherein the cellulose layer is applied by knife-coating, spraying, spin-coating, spray-drying, and/or dipping, optionally followed by drying.

Embodiment 17: The process according to any of embodiments 1 to 16, wherein the stable dispersion is a stable aqueous dispersion or a stable dispersion in a mixture of water and a water-miscible solvent.

Embodiment 18: The process according to any of embodiments 1 to 17, wherein the cellulose derivative comprises derivatization with ester and/or ether groups.

Embodiment 19: The process according to any of embodiments 1 to 18, wherein the cellulose derivative comprises derivatization with at least one functional group selected from carboxyl, carbonyl, sulfate, carboxymethyl, methyl, ethyl, silyl, acetyl, carbamate, and amino.

Embodiment 20: The process according to embodiment 18 or 19, wherein the cellulose derivative has a DS value of less than 0.5.

Embodiment 21: The process according to any of embodiments 1 to 20, wherein the cellulose layer comprises at least two non-identical celluloses and/or cellulose derivatives.

Embodiment 22: The process according to any of embodiments 1 to 21, wherein the cellulose was obtained from wood, annual plants, cotton, and/or waste paper.

Embodiment 23: The process according to any of embodiments 1 to 22, wherein the support comprises glass, paper, plastic, ceramic, and/or metal, preferably consists of glass, paper, plastic, ceramic, and/or metal.

Embodiment 24: A cellulose layer comprising an immobilized ligand, produced or producible by the process according to any of embodiments 1 to 23.

Embodiment 25: The cellulose layer according to embodiment 24, wherein the cellulose layer is transparent.

Embodiment 26: The cellulose layer according to embodiment 24 or 25, wherein the coated support is present in the form of a solid body, preferably in the form of a plate, film, membrane or bead.

Embodiment 27: The cellulose layer according to any of embodiments 24 to 26, wherein the coated support is a packaging material, a laboratory material, preferably a biochip or a multiwell plate, or a single-use article, preferably a urine cup, a syringe, a cannula, tubing, a tissue article, a swab, a breathing mask or part thereof, or an air filter.

Embodiment 28: A method for the detection of an analyte in a sample, comprising

(I) contacting the sample with a cellulose layer produced by the process according to any of embodiments 1 to 23 and/or with a cellulose layer according to any of embodiments 24 to 27, and

(II) detecting analytes interacting with the ligands present in the cellulose layer.

Embodiment 29: The method according to embodiment 28, wherein evaluation takes place visually or by imaging with fluorescence optics, luminescence optics or absorption optics, by scanning densitometry, or electrochemically.

Embodiment 30: The method according to embodiment 28 or 29, wherein the analyte is present in a sample of a body material.

Embodiment 31: The method according to any of embodiments 28 to 30, wherein the body material is a body fluid, preferably blood, plasma, serum or urine, or a gas, preferably exhaled air.

Embodiment 32: A device comprising a cellulose layer according to any of embodiments 24 to 29.

Embodiment 33: The device according to embodiment 32, wherein the device is a packaging material, a laboratory material, preferably a biochip or a multiwell plate, or a single-use article, preferably a urine cup, a syringe, a cannula, tubing, a tissue article, a swab, a breathing mask or part thereof, or an air filter.

Embodiment 34: A kit for the detection of at least one analyte, comprising at least one cellulose layer having an immobilized ligand and a device for sample collection.

Embodiment 35: The kit according to embodiment 34, wherein the cellulose layer is present on a support.

Embodiment 36: Use of a cellulose layer produced by the process according to any of embodiments 1 to 23 and/or of a cellulose layer according to any of embodiments 24 to 29 for detecting an analyte, preferably in medical diagnostics, (bio)analysis, environmental analysis, the agricultural, foodstuffs or packaging industry, process engineering or forensic medicine.

Embodiment 37: A process for producing transparent cellulose layers and the use thereof as multifunctional supports for ligands, characterized in that

a) the cellulose layer is applied to a support such as glass, paper, plastic, ceramic or metal,

b) the layer produced is immobilized with ligands,

c) the layer immobilized with ligands is used for detection for analytical purposes.

Embodiment 38: The process according to embodiment 37, wherein cellulose is applied to the support in the form of a stable aqueous dispersion or wherein cellulose is also dispersed in a mixture of water and a water-miscible solvent and can be applied to the support.

Embodiment 39: The process according to embodiment 37 to 38, wherein the cellulose dispersions are produced using cellulose from any possible source (wood, annual plants, cotton, waste paper) and wherein the cellulose dispersions may be produced using celluloses from cellulose derivatives having a DS value <0.5, wherein the cellulose derivatives may contain ether and/or ester functional groups such as carboxyl, carbonyl, sulfate, carboxymethyl, methyl, ethyl, silyl, acetate, carbamate, and amino.

Embodiment 40: The process according to embodiments 37 to 39, wherein the dispersions comprising cellulose or cellulose derivatives have a solids content (mass fraction) of between 0.05% and 5% (w/w) and the ingredients have particle sizes <600 nm.

Embodiment 41: An application of a homogeneous cellulose layer, which may also consist of a mixture of a plurality of different dispersions comprising cellulose or cellulose derivatives according to embodiments 1 to 4, produced by knife-coating, spraying, spin-coating, dipping or spray-drying or by a combination of said methods.

Embodiment 42: A coated support produced by means of the process according to embodiments 37 to 41, which may be solid, flexible, planar, beads, films, and membranes, packaging materials of any kind.

Embodiment 43: The process according to embodiments 37 to 42, wherein the cellulose layers produced are used in medical diagnostics, (bio)analysis, environmental analysis, the agricultural, foodstuffs or packaging industry, process engineering or forensic medicine and the lifestyle sector.

Embodiment 44: The process according to embodiments 37 to 43, wherein the ligands used are all molecules with which selective binding of analytes from a sample can be achieved and wherein the binding thereof can be detected subsequently after washing (heterogeneous assay) or subsequently/simultaneously without washing (homogeneous assay).

Embodiment 45: The process according to embodiments 37 to 43, wherein the selection of the ligands is strongly dependent on the intended use. Ligands can be understood as meaning proteins, peptides, nucleic acids, oligonucleotides, carbohydrates, lipids or fats, in particular antibodies, antigens, hormones, glycolipids, phospholipids, glycoproteins, phosphoproteins, recombinant proteins, native proteins, autoantigens, allergens, and cells. The term also encompasses molecules present in living systems or killed systems where these systems are wholly or partially immobilized.

Embodiment 46: The process according to embodiments 37-45, characterized in that evaluation takes place visually or by imaging with fluorescence optics, luminescence optics or absorption optics, by scanning densitometry or electrochemically.

Embodiment 47: A process for producing transparent cellulose layers and the use thereof as multifunctional supports for ligands, characterized in that

a) the cellulose layer is applied to a flexible or non-flexible support such as glass, paper, plastic, ceramic or metal,

b) the layer, after drying, is immobilized with ligands,

c) the layer immobilized with ligands is used for detection for analytical purposes.

Embodiment 48: The process according to embodiment 47, wherein cellulose is applied to the support in the form of a stable aqueous dispersion.

Embodiment 49: The process according to embodiment 47 or 48, wherein a homogeneous cellulose layer is produced by knife-coating, spraying, spin-coating, dipping or spray-drying or by a combination of said methods.

Embodiment 50: The process according to any of embodiments 47 to 49, wherein the cellulose dispersions are produced using cellulose from any possible source (wood, annual plants, cotton, waste paper).

Embodiment 51: The process according to any of embodiments 47 to 50, wherein the cellulose derivatives contain ether and/or ester functional groups such as carboxyl, alkyl and aryl, sulfate, phosphate, carbonyl, carboxymethyl, acetate, carbamate, amino, ammonium, silyl groups, wherein the degree of substitution DS is <0.5.

Embodiment 52: The process according to any of embodiments 47 to 51, wherein the cellulose layer may also a mixture of a plurality of different dispersions comprising cellulose or cellulose derivatives.

Embodiment 53: The process according to any of embodiments 47 to 52, wherein the dispersions comprising cellulose or cellulose derivatives have a solids content (mass fraction) of between 0.05% and 5% (w/w).

Embodiment 54: The process according to any of embodiments 47 to 53, wherein the dispersions comprising the cellulose or cellulose derivatives are characterized in that the ingredients mentioned have particle sizes <600 nm, preferably in the range between 300-100 nm.

Embodiment 55: The use of a cellulose layer produced according to any of embodiments 47 to 54 in medical diagnostics, (bio)analysis, environmental analysis, agriculture and the foodstuffs industry, the packaging industry, or in forensic medicine.

Embodiment 56: The process according to any of embodiments 47 to 54, wherein the cellulose layer is applied to solid, flexible, planar, cylindrical or ellipsoidal supports, such as beads, tubes, pipes, films or membranes.

Embodiment 57: The use of a cellulose layer produced according to any of embodiments 47 to 54 for coating packaging materials of any kind, laboratory materials such as biochips, microtiter plates or medical single-use articles such as urine cups, syringes and cannulas, tubing, tissue articles, swabs, breathing masks or parts thereof, or air filters.

Embodiment 58: The subject matter of any of embodiments 47 to 57, wherein the ligands are all molecules with which selective binding of analytes from a sample can be achieved and wherein the binding thereof can be detected subsequently after washing or subsequently/simultaneously without washing, wherein the choice of ligands is strongly dependent on the intended use, such as use in medical diagnostics, bioanalysis, environmental analysis or forensic medicine.

Embodiment 59: The subject matter of embodiment 58, wherein ligands are for example proteins, peptides, nucleic acids, oligonucleotides, carbohydrates or fats, preferably antibodies, antigens, hormones, glycolipids, phospholipids, glycoproteins, phosphoproteins, recombinant proteins, native proteins, autoantigens, allergens or allergen complexes, or cells, preferably molecules present in living systems or killed systems where these systems are wholly or partially immobilized.

Embodiment 60: The subject matter of any of embodiments 47 to 59, characterized in that evaluation takes place visually or by imaging with fluorescence optics, luminescence optics or absorption optics, by scanning densitometry or electrochemically.

Embodiment 61: The subject matter of any of embodiments 47 to 60, wherein the coated support furnished with ligands includes reagents for washing and/or detection.

Embodiment 62: The use of a cellulose layer produced according to any of embodiments 47 to 54 for analysis of a sample matrix from human or veterinary diagnostics;

in particular for analysis of urine, blood, serum, respiratory gas, sweat, or of swabs from feces, throat, nose and relevant surfaces from the human or veterinary sector.

Embodiment 63: The use of a cellulose layer produced according to any of embodiments 47 to 54 for analysis of a sample matrix from environmental science or life sciences, particularly in the analysis of freshwater and drinking water, process water and waste water, soil, air and exhaust air.

Embodiment 64: A device comprising a cellulose layer produced according to any of embodiments 47 to 54 configured for analysis of a sample from human or veterinary diagnostics; in particular for analysis of urine, blood, serum, respiratory gas, sweat, or of swabs from feces, throat, nose and relevant surfaces from the human or veterinary sector.

Embodiment 65: A device comprising a cellulose layer produced according to any of embodiments 47 to 54 configured for analysis of a sample from environmental science or life sciences, particularly in the analysis of freshwater and drinking water, process water and waste water, soil, air and exhaust air.

All publications cited in this description are hereby incorporated by reference into the disclosure in respect of their entire disclosure content.

The following examples serve solely to illustrate the invention. They are not to be understood as restricting the invention or the included claims.

The present invention relates to the production of transparent cellulose films from aqueous cellulose dispersions and to the use thereof as multifunctional supports for tests in medical diagnostics, food and environmental analysis, and other areas in which analytical problems arise. The transparent films may be applied to various flexible and non-flexible supports made of plastic, glass, ceramic, metal, and paper, and have been shown to have storage stability.

### Example 1: Dispersions

For the following examples, various aqueous cellulose dispersions were used. First, 0.8 g of the cellulose or of the respective cellulose derivative was weighed out and made up to 100 g with deionized water. The samples were then treated with an Ultra-Turrax at approx. 20 000 rpm for 15 min. After being allowed to rest for 15 min, the procedure is repeated. This is followed by a two-stage treatment in a high-pressure homogenizer. This consists of the performance of 6 cycles in a 200 μm cell at 500 bar and 12 cycles in a 50 μm cell at 1000 bar. Homogeneous aqueous dispersions having a storage stability of more than 3 years are obtained.

All the listed dispersions were in each case transferred to a commercial slide and dispersed homogeneously on the surface with a doctor blade.

TABLE 1
Dispersions

| Sample | Cellulose/cellulose derivative | Slide |
|---|---|---|
| Dispersion 1 | Cellulose (DP = 380) | Sl D1 |
| Dispersion 2 | Oxidized cellulose | Sl D2 |
| | (carboxyl content = 22 mol. eq./100 g) | |
| Dispersion 3 | Oxidized cellulose | Sl D3 |
| | (carboxyl content = 48 mol. eq./100 g) | |
| Dispersion 4 | Oxidized cellulose | Sl D4 |
| | (carboxyl content = 66 mol. eq./100 g) | |
| Dispersion 5 | Carboxymethylated cellulose (DS = 0.1) | Sl D5 |
| Dispersion 6 | Cellulose sulfate (DS = 0.05) | Sl D6 |
| Dispersion 7 | Silylcellulose (DS = 0.3) | Sl D7 |
| Dispersion 8 | Methylcellulose (DS = 0.5) | Sl D8 |

### Example 2: Coating of Glass Slides and Array Production

Commercial slides were with the dispersions listed in Table 1 and coated using a doctor blade as described in Example 1.

For the production of arrays, various ligand molecules, such as DNA, peptides, and proteins, are dissolved in liquids and applied in the form of tiny droplets (spots) to the described surfaces (microarray technology). The ligand molecules are able to bind to the surface specifically via the reactive surface coating. None of the surfaces were preactivated.

All microarrays could be processed readily. They withstood multiple washing, blocking, and incubation of the analytes without the film layer becoming detached.

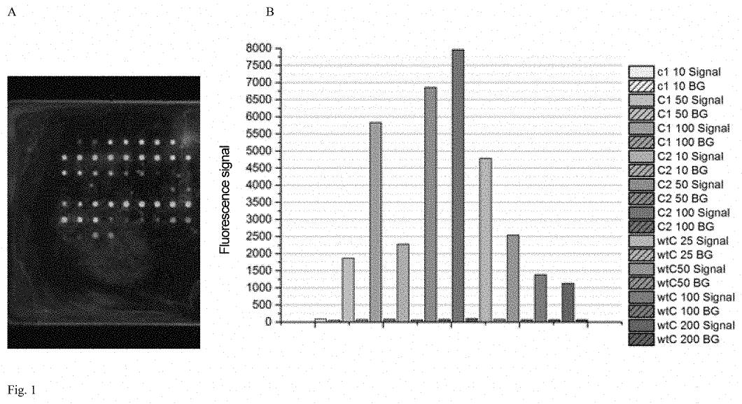

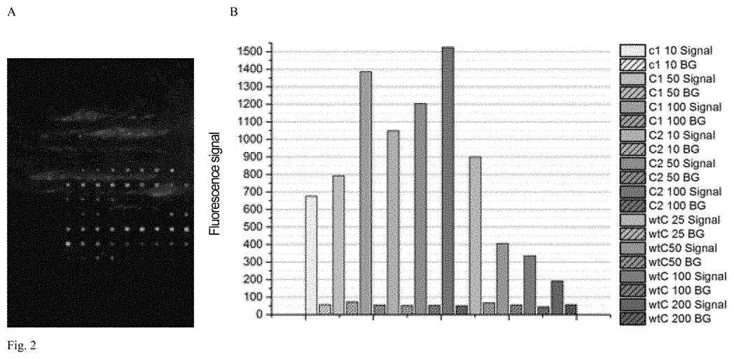

In microarray technology, many proteins are labeled with Cy5 dyes, which are then read at 635 nm. This is not possible with commercial nitrocellulose slides on account of the high level of autofluorescence. It is a clear advantage of the new coating that the user is able to use commercially available microarray readers having red (standard Cy5) and green (standard Cy3) lasers. A comparison of the signal-background intensities is shown in FIGS. 1 and 2, FIG. 1: Cellulose layer, FIG. 2: Epoxy slide.

### Example 3: Diagnostic Protein Chip

According to the basic principle of the Western blot, peptides and proteins as ligand molecules are bound and detected in varying concentrations on the described surfaces. The great diversity in the chemical properties of proteins (acidic/basic, hydrophilic/hydrophobic, structural modifications) makes these molecules sensitive to the properties of the coated support. In contrast to DNA chips, the protein or peptide analytes undergo secondary detection with labeled ligands (antibodies).

### Example 4: Production of the Layers Using Different Methods and Determination of the Layer Thickness by AFM (Bruker Dimension Icon)

Transparent layers on commercial glass supports (microscope slides) were produced using different methods. For this purpose, an aqueous cellulose dispersions (0.71%, w/w) were applied to the support using the respective method and the layer thickness then determined using an AFM (atomic force microscope) from Bruker (Dimension Icon model). The results are shown in Table 2. After the slides had been coated, they were dried in a circulating-air drying cabinet at 45° C. for 5 minutes. The layer thicknesses were measured at three points on the slides. The value in the table corresponds to the arithmetic mean.

TABLE 2

| Method | Layer thickness [μm] |
|---|---|
| Dropping with a Pasteur pipette | 3.2 |
| Knife-coating | 2.2 |
| Spin-coating | 0.05 |
| Dipping | 0.03 |

### Example 5: Production of Layers as a Function of the Cellulose Content

To investigate the dependence of the layer thickness on the cellulose content of the dispersions, various slides are dispersed on the support using a doctor blade. The results are shown Table 3.

TABLE 3

| Cellulose content (% w/w) | Layer thickness [μm] |
|---|---|
| 0.71 | 2.2 |
| 0.88 | 2.5 |
| 1.20 | 3.6 |

### Example 6

Motivation
Trend: Multiparameter analysis->simultaneous determination of a plurality of analytes in one measurement run
More complex analytical information after just one laboratory investigation
Faster, better, and more cost-efficient analysis by virtue of miniaturization and parallel determination of a plurality of analytes in just one test
Optimized materials and optimized material surfaces are key, irrespective of the employed technologies.
The requirement for support materials for use in surface-bound analyses is the ability to permit high loading densities and optimal functionality in the context of the respective problem.

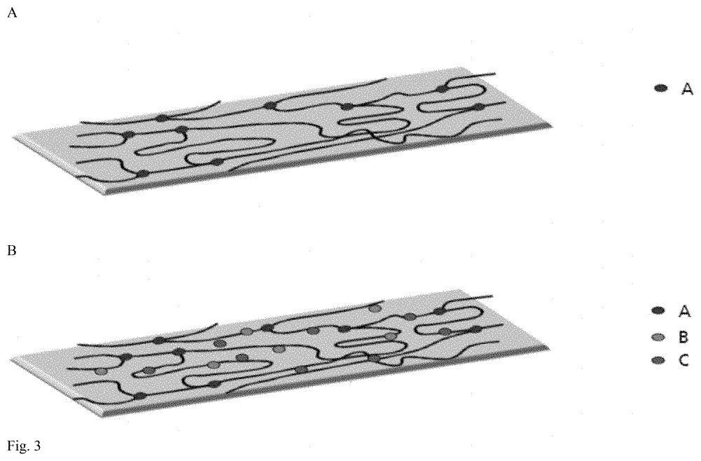

Structure of a solid-phase test for the detection of an analyte (FIG. 3A, B)
Support: Glass, plastic, paper, metal
Coating: (Chemical) functionalities for the specific binding of ligands (DNA, peptides, proteins, etc.)
Ligands
Detection systems: Dyes (UV, fluorescence, luminescence), metal nanoparticles, latex particles, etc.

A coating having one functionality (FIG. 3A), generation of a coating having different functional groups that serve to immobilize the ligands (FIG. 3B).

Technical execution of multiparametric tests, using peptide and protein chips by way of example
Different ligands for each analyte, in parallel
Or in each case one ligand for multiple analytes, e.g. for screening experiments

Aim—High binding capacity with low nonspecific binding
Surface has no effect on the structure of the peptide or protein
Hindrance of method by unsuitable surface that is in direct contact with the environment
High sensitivity, relatively high signal intensity—porous materials and fibers

Proteome analysis on nitrocellulose slides

Pros
Stable protein structure on the surface is maintained
High binding affinity/binding capacity of the spotted proteins
Porous surface
Stability at room temperature
Long-term stability

Cons
High autofluorescence
Poor wetting (hydrophobic surface)
Subsequent functionalization not possible

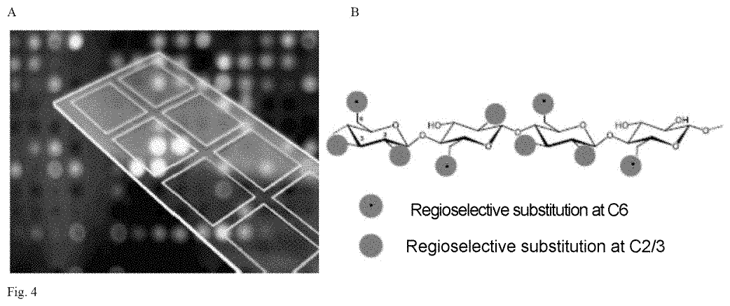

Proteome analysis on cellulose slides (FIG. 4)

Pros
Stable protein structure on the surface is maintained
High binding affinity/binding capacity of the spotted proteins
Porous surface
Stability at room temperature
Long-term stability
Transparent, good film former
Good wetting (hydrophilic surface)
Subsequent functionalization possible

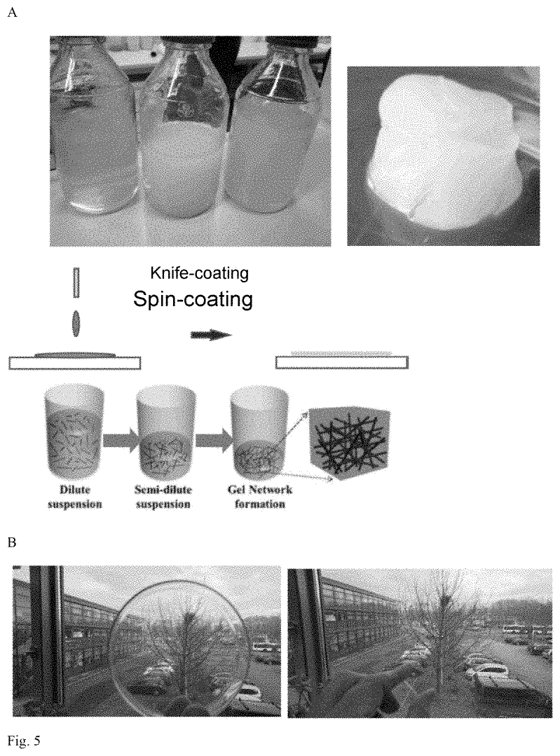

FIG. 5 shows a schematic exemplary representation of the production of layers of the invention and examples for the transparency properties of the cellulose layers of the invention.

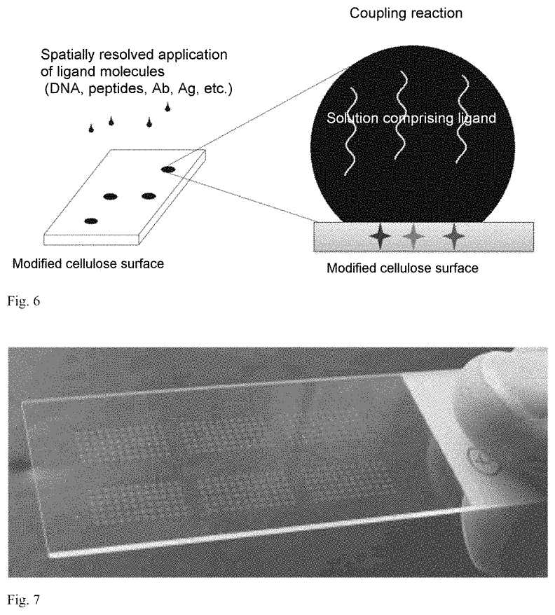

Result of coating (FIGS. 6, 7)
Modified cellulose as a film on a solid support (without subsequent functionalization)
Transparent film
Optically readable
Micropore structure
Modification of layer thickness
Stability toward external influences (buffer salts, pH, humidity, temperature)
No surface activation necessary
No blocking

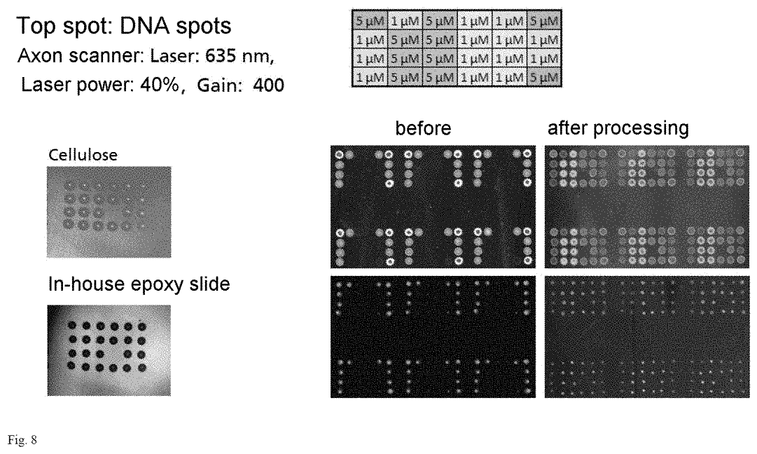

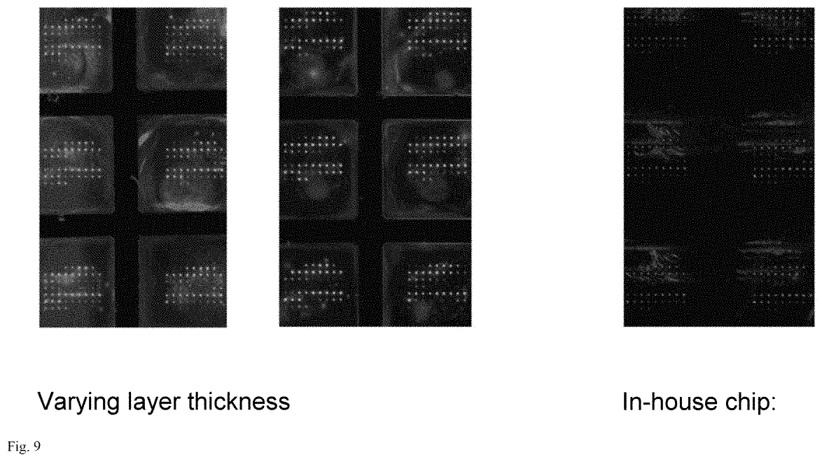

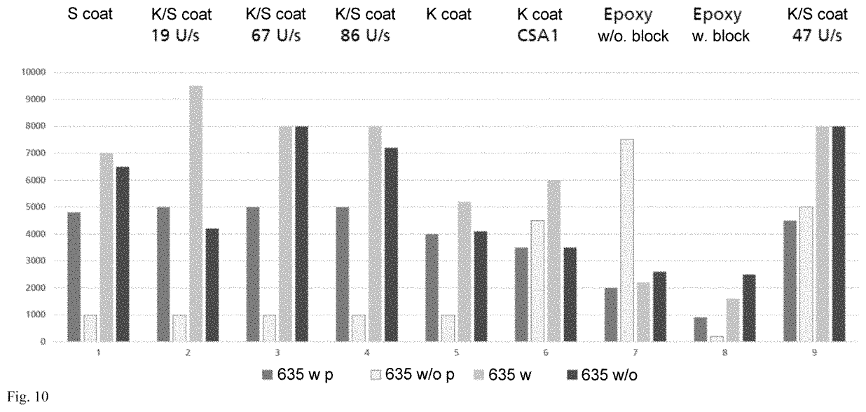

FIG. 8 shows spot morphology in a comparison of cellulose and epoxy slide coatings, FIG. 9 shows a stress test of the surface, comparing different layer thicknesses of cellulose slides with epoxy slide. FIG. 10 shows an example of immobilization and detection of proteins and peptides on a support.

1. A process for producing a cellulose layer for the detection of at least one analyte, comprising:
(i) producing a cellulose layer by applying a stable dispersion of cellulose and/or a cellulose derivative to a suitable support, and
(ii) immobilizing at least one ligand on the cellulose layer.

2. The process as claimed in claim 1, wherein the cellulose layer is present on a support.

3. The process as claimed in claim 1, wherein the ligand selectively binds the at least one analyte.

4. The process as claimed in claim 1, wherein the ligand is a polypeptide, a polynucleotide, a carbohydrate, or a fat.

5. The process as claimed in claim 1, wherein the ligand is an antibody, a hormone, a glycolipid, a phospholipid, a glycoprotein, or a phosphoprotein.

6. The process as claimed in claim 1, wherein the ligand is or comprises a recombinant protein, a native protein, an autoantigen, an allergen and/or a cell.

7. The process as claimed in claim 1, wherein a multiplicity of non-identical ligands are immobilized on the cellulose layer.

8. The process as claimed in claim 1, wherein immobilization takes place in a spatially structured manner.

9. The process as claimed in claim 1, wherein the ligand is covalently bonded to the cellulose layer.

10. The process as claimed in claim 1, wherein the cellulose layer obtained is transparent.

11. The process as claimed in claim 1, wherein the stable dispersion has a solids content of between 0.05% (w/w) and 5% (w/w).

12. The process as claimed in claim 1, wherein the cellulose and/or the cellulose derivative has a particle size of not more than 600 nm.

13. The process as claimed in claim 1, wherein the cellulose derivative has derivatization with ester and/or ether groups.

14. The process as claimed in claim 1, wherein the cellulose layer comprises at least two non-identical celluloses and/or cellulose derivatives.

15. The process as claimed in claim 1, wherein at least two non-identical celluloses and/or cellulose derivatives are dispersed together before application.

16. A cellulose layer produced by the process as claimed in claim 1, comprising an immobilized ligand.

17. The cellulose layer as claimed in claim 16, wherein the suitable support is a packaging material, a laboratory material, or a single-use article.

18. A method for the detection of an analyte in a sample, the method comprising:
contacting the sample with a cellulose layer produced by the process as claimed in claim 1, such that analyte present in the sample interacts with the at least one ligand in the cellulose layer, and
detecting the interaction of the analytes with the at least one ligand present in the cellulose layer.

19. The method as claimed in claim 18, wherein detecting the analyte is performed visually or by imaging with fluorescence optics, luminescence optics or absorption optics, by scanning densitometry or electrochemically.

20. The method as claimed in claim 18, wherein the sample is selected from the group consisting of blood, plasma, serum, urine, and exhaled air.

21. A device comprising a cellulose layer as claimed in claim 16.

22. The device as claimed in claim 21, wherein the device is a packaging material, a laboratory material, or a single-use article.

23. A kit for the detection of at least one analyte, comprising:
at least one cellulose layer having an immobilized ligand, said layer being produced by the process as claimed in claim 1, and
a device for sample collection.

24. The kit as claimed in claim 23, wherein the cellulose layer is present on a support.

25. (canceled)
